(19) United States
(12) Patent Application Publication
(10) Pub. No.: US 2023/0331650 A1
(43) Pub. Date: Oct. 19, 2023

(54) METHOD AND PLANT FOR PREPARING DIMETHYL ETHER

(71) Applicant: Linde GmbH, Pullach (DE)

(72) Inventor: Andreas PESCHEL, Wolfratshausen (DE)

(21) Appl. No.: 18/042,690

(22) PCT Filed: Jun. 22, 2021
(86) PCT No.: PCT/EP2021/025224

§ 371 Date: Feb. 23, 2023

(30) Foreign Application Priority Data
Aug. 31, 2020 (EP) 20020393.3

(51) Int. Cl. C07C 41/09; C07C 41/42; C07C 29/151; B01D 53/14; B01D 53/00; B01D 3/14

(52) U.S. Cl. CPC C07C 41/09; C07C 41/42; C07C 29/151; B01D 53/1425; B01D 53/1475; B01D 53/1487; B01D 53/002; B01D 3/143; B01D 2252/103; B01D 2257/70; B01D 2257/504; B01D 2257/502; B01D 2257/108

(57) ABSTRACT

A method for preparing dimethyl ether (DME) from synthesis gas, wherein an input, which is formed using shifted and/or non-shifted synthesis gas, undergoes a catalytic conversion, thereby forming a product stream. The product stream undergoes a first separation, wherein a gas mixture is formed by at least partial separation of methanol and/or water from the product stream, and the gas mixture is partially condensed at a first pressure level by means of cooling from a first to a second temperature level. A portion of the gas mixture remaining in gaseous form at the second temperature level is washed in an absorption column with a return predominantly containing dimethyl ether, wherein a dimethyl ether product is formed using the portion of the gas mixture condensed during cooling.

The invention relates to a method for preparing dimethyl ether (DME) and to a plant for carrying out such a method and to a plant for the separation processing of an intermediate product of the method.

### PRIOR ART

Dimethyl ether (DME) is the structurally simplest ether. Dimethyl ether contains two methyl groups as organic moieties. Dimethyl ether is polar and is conventionally used in liquid form as solvent. Dimethyl ether can also be used as coolant and replace conventional chlorofluorocarbons.

Recently, dimethyl ether is increasingly used as a substitute for fuel gas (liquefied gas) and conventional fuels such as diesel. Due to its comparatively high cetane number of 55 to 60, conventional diesel engines, for example, only have to be modified slightly for operation with dimethyl ether. Dimethyl ether burns comparatively cleanly and without soot formation. If dimethyl ether is prepared from biomass, it is considered to be a so-called biofuel and can therefore be marketed at lower tax rates.

Dimethyl ether can be produced either directly from methanol or indirectly from natural gas or biogas. In the latter case, the natural gas or biogas is first reformed to form synthesis gas. Synthesis gas is a gas mixture which contains at least carbon monoxide and hydrogen in changing portions, but which together make up the predominant part of the gas mixture. Synthesis gas can also be obtained by means of other methods, for example by pyrolysis of coal, oil, carbonaceous waste, biomass or other carbon-containing starting materials, by dry reforming of natural gas with carbon dioxide, by steam reforming of natural gas, by autothermal reforming (ATR) of natural gas, by partial oxidation (POX) of hydrocarbons, in particular natural gas or methane, or combinations of the aforementioned methods. The synthesis gas is then converted either in a two-stage reaction into methanol and then into dimethyl ether or in a single-stage reaction directly into dimethyl ether.

The synthesis of dimethyl ether from synthesis gas is thermodynamically and economically more advantageous than synthesis from methanol.

The present invention relates in particular to the single-stage synthesis of dimethyl ether, wherein a “single-stage” synthesis is understood to mean a synthesis in which all reactions proceed in the same reactor. The single-stage synthesis of dimethyl ether is known, for example, from U.S. Pat. Nos. 4,536,485 A and 5,189,203 A. Hybrid catalysts are conventionally used in this case. The reaction is exothermic and typically takes place at a temperature of 200 to 300° C. and at a pressure of 20 to 100 bar.

For single-stage synthesis of dimethyl ether, normally upright tubular reactors are used, which are charged from below with pressurized and heated synthesis gas. A product stream obtained in the tubular reactor is withdrawn from the top, cooled and fed to a separation process.

Besides dimethyl ether, the product stream contains unreacted components of the synthesis gas and further reaction products. Typically, the product stream comprises, in addition to dimethyl ether, at least methanol, water, carbon dioxide, carbon monoxide and hydrogen, and, in lower amounts, methane, ethane, organic acids and higher alcohols.

In a gas mixture formed from the product stream, carbon dioxide and components which boil more easily than carbon dioxide, such as hydrogen and carbon monoxide, are therefore typically present in addition to dimethyl ether. These must be at least partially separated out to obtain dimethyl ether conforming to specifications. Methods used for this purpose, however, prove to be unsatisfactory, in particular in terms of energy.

To obtain dimethyl ether from the product stream, the latter must be cooled to temperatures significantly below 0° C. It may be necessary here to separate out relatively large amounts of methanol and water before cooling. However, WO 2015/104290 A1 also discloses methods in which such a separation is not necessary.

An improved recycling concept for unreacted synthesis gas is to be specified.

### DISCLOSURE OF THE INVENTION

This object is achieved by a method for preparing dimethyl ether (DME), a plant for the separation processing of a gas mixture and a plant for carrying out such a method, having the features of the respective independent claims. Advantageous developments of the invention form the subject matter of the dependent claims and the description below. Before explaining the features and advantages of the present invention, the principles and the terms used are explained.

A fluid (the term “fluid” is also used below for short for corresponding streams, fractions, etc.) is “derived” from another fluid (which is also referred to as the starting fluid) or “formed” from such a fluid when it has at least some components contained in the starting fluid or obtained therefrom. A fluid derived or formed in this sense can be obtained or formed from the starting fluid by separating or branching off a portion or one or more components, enriching or depleting with respect to one or more components, chemically or physically reacting one or more components, heating, cooling, pressurizing, and the like. A stream can also be simply “formed”, for example, by being drawn off from a storage tank.

Fluids can, in the terminology used herein, be rich or low in one or more contained components, wherein “rich” can refer to a content of at least >50%, 75%, 90%, 95%, 99%, 99.5%, 99.9%, 99.99% or 99.999%, and “low” can refer to a content of at most 10%, 5%, 1%, 0.1%, 0.01% or 0.001% on a molar, weight, or volume basis. In the terminology used herein, they can be enriched with or depleted of one or more components, wherein these terms relate to a corresponding content in a starting fluid from which the fluid was formed. The fluid is “enriched” if it contains at least 1.1 times, 1.5 times, 2 times, 5 times, 10 times, 100 times or 1000 times the content, and “depleted” if it contains at most 0.9 times, 0.5 times, 0.1 times, 0.01 times or 0.001 times the content of a corresponding component, in relation to the starting fluid. A fluid containing “predominantly” one or more components contains said one or more components in a percentage of at least >50%, 75%, 90%, 95%, 98% or 99% or is rich in them.

The terms “pressure level” and “temperature level” are used below to characterize pressures and temperatures, whereby it is intended to be expressed that pressures and temperatures do not need to be used in the form of exact pressure or temperature values in order to realize an inventive concept. However, such pressures and temperatures typically fall within certain ranges that are, for example, ±1%, 5%, 10%, 20% or even 50% around an average. Different pressure levels and temperature levels may be in disjoint ranges or in ranges which overlap one another. In particular, pressure levels, for example, include unavoidable or expected pressure losses, for example due to cooling effects. The same applies to temperature levels. The pressure levels indicated here in bar are absolute pressures.

In the terminology used herein, a “distillation column” is a separation unit which is configured to separate at least partially a substance mixture (fluid) provided in gaseous or liquid form or in the form of a two-phase mixture with liquid and gaseous portions, possibly also in the supercritical state, i.e., to produce pure substances or substance mixtures from the substance mixture, which are enriched or depleted in relation to the substance mixture with respect to at least one component or are rich or low in the sense explained above. Distillation columns are well-known from the field of separation technology.

Distillation columns are typically designed as cylindrical metal containers which are equipped with fittings, such as sieve trays or ordered or disordered packings. A distillation column is characterized, inter alia, in that a liquid fraction is deposited in its lower region, also referred to as the bottom. This liquid fraction, which is referred to here as the bottom liquid, is heated in a distillation column by means of a bottom evaporator, so that some of the bottom liquid is continuously evaporated and rises in gaseous form in the distillation column. A distillation column is also typically provided with a so-called top condenser, into which at least some of a gas mixture enriched in an upper region of the distillation column or a corresponding pure gas, referred to here as top gas, is fed, liquefied in part to form a condensate, and provided as a liquid return at the top of the distillation column. Some of the condensate obtained from the top gas can be used elsewhere.

In contrast to a distillation column, an “absorption column” typically does not have a bottom evaporator. Absorption columns are also generally known from the field of separation technology. Absorption columns are used for absorption in the phase counter-current and are therefore also referred to as counter-current columns. During absorption in the countercurrent, the releasing gas phase flows upwards through an absorption column. The receiving solution phase, provided from above and withdrawn at the bottom, flows towards the gas phase. The gas phase is “washed” with the solution phase. Also typically provided in a corresponding absorption column are built-in components which ensure a step-by-step (trays, spray zones, rotating plates, etc.) or continuous (random filling of filling material, packings, etc.) phase contact. At the top of such an absorption column, a gaseous fluid is obtained which can be drawn off therefrom as a “top product.” A liquid which can be drawn off as “bottom product” is deposited in the bottom of the absorption column. The gas phase is depleted in the absorption column with respect to one or more components, which pass into the bottom product.

For the design and specific configuration of distillation columns and absorption columns, reference is made to relevant textbooks (see, for example, Sattler, K.: Thermische Trennverfahren: Grundlagen, Auslegung, Apparate, 3rd edition 2001, Weinheim, Wiley-VCH).

Where a “synthesis” of dimethyl ether is mentioned for short below, this is understood to mean a process in which a synthesis gas or syngas-containing input, i.e., a gas mixture which contains at least carbon monoxide and hydrogen in suitable portions, is converted into a corresponding product stream containing dimethyl ether. The portion of hydrogen in the synthesis gas can be increased, for example, using a water-gas shift reaction. In this case, carbon monoxide present in the synthesis gas reacts with water (steam) added for this purpose to form carbon dioxide, which can be separated out if necessary. A synthesis gas aftertreated in this way is also referred to as a shifted synthesis gas. A corresponding product stream of the synthesis of dimethyl ether contains not only dimethyl ether but also further compounds, due to the incomplete reaction and due to the occurrence of side reactions during the synthesis of dimethyl ether, in particular depending on the characteristics of the catalysts used and the respective contents of the components of the synthesis gas. These are at least methanol, water, carbon dioxide, carbon monoxide and hydrogen, but typically also lower amounts of methane, ethane, organic acids and higher alcohols. Said further compounds have to be separated out, as mentioned. The separation is carried out on the one hand to enable subsequent separation steps and on the other hand to obtain dimethyl ether in the required purity, i.e., “conforming to specification.”

### Advantages of the Invention

In the method according to the invention, as explained, a gas mixture is separation processed which is formed from a product stream of a reactor for the synthesis of dimethyl ether from synthesis gas and which contains at least dimethyl ether, carbon dioxide and at least one further component which boils more easily than carbon dioxide. Such components which boil more easily than carbon dioxide can in particular be components such as carbon monoxide and hydrogen. As mentioned, other components, which likewise boil more easily than carbon dioxide, for example methane, are likewise contained in a smaller amount in such a gas mixture.

To increase the yield, unreacted synthesis gas is, according to the invention, at least partially conveyed back to the synthesis as a recycling stream and undergoes the synthesis again.

The catalyst used in the synthesis is sensitive to carbon dioxide concentrations exceeding 8% or also 4% of the amount of the input mixture. Since the amount of carbon dioxide formed in the synthesis depends on the composition of the input mixture, shifted and non-shifted synthesis gas in the required ratio is mixed according to the invention. For example, a ratio of hydrogen to carbon monoxide is set in the range from 1.15 to 1.5. Shifted synthesis gas is in particular rich in hydrogen, non-shifted is in particular rich in carbon monoxide. The smaller the ratio of the portions of hydrogen to carbon monoxide in the input mixture, the more carbon dioxide is formed during the synthesis. This can be understood easily from the following highly simplified reaction equations:

3 H₂+3 CO→DME+CO₂ (H₂:CO=1)

4 H₂+2 CO→DME+H₂O (H₂:CO=2)

If carbon dioxide is now removed from the recycling stream before it is returned to the synthesis, the unconsumed constituents of the input can be conveyed back to the synthesis, without loading the catalyst with carbon dioxide that is harmful or disruptive for this, even if the ratios of hydrogen to carbon monoxide are low. Therefore, the method can be operated with a lower portion of shifted synthesis gas, which is economically advantageous, since a corresponding plant for the shifting can be smaller or even completely eliminated. The method can thus be operated with an inexpensive non-shifted synthesis gas in a higher portion or entirely.

Advantageously, not all the unconsumed synthesis gas is conveyed back to the synthesis as the recycling stream, since impurities, which can be introduced into the method via the fresh input, for example, are enriched by such a closed circulation system. Such impurities can be, for example, argon, nitrogen, oxygen, helium or other inert gases and, for example, alcohols, aldehydes, ketones, esters, ethers, olefins, alkanes and other hydrocarbons, which can result as by-products from the synthesis or the synthesis gas provision.

A component which “boils more easily than carbon dioxide” has a lower boiling point than carbon dioxide. It should be pointed out at this juncture that carbon dioxide can also be present in the liquid phase at the pressure levels used according to the invention (the “first” pressure level explained below is above the triple point of carbon dioxide).

According to the invention, the gas mixture at the first pressure level is cooled from a first temperature level to a second temperature level. This can advantageously take place via one or more intermediate temperature levels and with deposition of one or more condensates out of the gas mixture, but single-stage cooling is also possible.

If condensates are deposited beforehand, these predominantly contain dimethyl ether and carbon dioxide, since the gas mixture used according to the invention is low in higher-boiling components such as methanol and water due to a prior separation of methanol and water from the product stream. An arrangement as shown in FIG. 2, for example, can be used for this. In particular, the gas mixture subjected to the cooling can be free or largely free of methanol and water. In the case of streams containing water and/or methanol, however, care must be taken to ensure that neither methanol nor water alone are contained in a corresponding gas mixture. Water without an additional portion of methanol would freeze at the low temperatures used because the antifreeze effect of the methanol would be absent. Conversely, however, “dry” methanol would also lead to disadvantages because it could damage the heat exchangers used. In addition to methanol, dimethyl ether is also suitable as antifreeze for the water-containing streams in each case alternatively or additionally.

A portion of the gas mixture remaining in gaseous form at the second temperature level is washed in an absorption column with a return predominantly containing dimethyl ether, obtaining a top product and a bottom product. This serves to wash out carbon dioxide still present in the portion of the gas mixture remaining in gaseous form into the bottom product, so that the top product is largely free of carbon dioxide if possible. The top product consists predominantly of the at least one component which boils more easily than carbon dioxide at the first pressure level. The top product preferably does not contain any carbon dioxide or contains at most small amounts, i.e., is at least low in carbon dioxide in the sense explained above. According to the invention, the top product is at least partially conveyed from the absorption column back into the synthesis. It is therefore important, as explained in the introduction, that as little carbon dioxide as possible is present in this recycling stream, since this can damage the catalyst used in the synthesis. The separation of carbon dioxide from this recycling stream according to the invention therefore enables longer catalyst lifetimes or use cycles.

In some embodiments, it can also be provided to set a certain portion of carbon dioxide in the recycling stream in a targeted manner, for example such that a portion of approximately 2% to approximately 6% of carbon dioxide is present on entry into the synthesis, which can have a positive effect on the reaction rate in the synthesis.

It may also be advantageous to feed a fresh input into the synthesis via the absorption column in order to adjust the carbon dioxide content in the input stream in a targeted manner. In such a case, the fresh input is dried upstream of the absorption column.

The return predominantly containing dimethyl ether is formed according to the invention from a portion of the gas mixture deposited in liquid form during cooling to the second temperature level. A dimethyl ether carbon dioxide distillation column is used for this purpose. If one or more condensate(s) are deposited during cooling, these or streams formed from these are at least partially fed into the dimethyl ether carbon dioxide distillation column. The bottom product from the absorption column or a stream formed therefrom can also be at least partially fed into the dimethyl ether carbon dioxide distillation column.

In this case, a “dimethyl ether carbon dioxide distillation column” is understood to mean a distillation column which is designed and operated in such a way that, from fluids containing dimethyl ether and carbon dioxide, fluids can be obtained therein which are on the one hand enriched with dimethyl ether and on the other hand enriched with carbon dioxide and depleted of the other component in each case. The person skilled in the art selects the specific configuration of a dimethyl ether carbon dioxide distillation column (such as, for example, the type and number of fittings) and the operating conditions (such as, for example, operating pressure, heating and cooling fluids in the bottom evaporator and top condenser, etc.) in particular on the basis of the differences in boiling point between dimethyl ether and carbon dioxide.

An essential aspect of the present invention is the suitable setting of the operating conditions of the dimethyl ether carbon dioxide distillation column. These are advantageously selected such that a top gas which predominantly contains carbon dioxide and is drawn off from the top of the dimethyl ether carbon dioxide distillation column can be liquefied above the melting temperature of carbon dioxide. A correspondingly obtained condensate should be available in an amount sufficient to be used as a return for the dimethyl ether carbon dioxide distillation column. A portion of the bottom liquid of the dimethyl ether carbon dioxide distillation column is made available as a return for the absorption column, as already explained in part.

The dimethyl ether carbon dioxide distillation column is advantageously operated in such a way that a top gas predominantly containing carbon dioxide forms at the top, and a bottom liquid enriched with dimethyl ether forms at the bottom. The bottom liquid contains predominantly dimethyl ether when the fluids fed into the dimethyl ether carbon dioxide distillation column (for example the described condensate(s) and the bottom product from the absorption column) predominantly contain carbon dioxide and dimethyl ether.

If the latter also contain water and methanol, in particular in a small portion, they likewise pass into the bottom liquid of the dimethyl ether carbon dioxide distillation column.

The invention thus enables a reduction in the portion of carbon dioxide in the unreacted synthesis gas conveyed back into the synthesis by using the dimethyl ether rich liquid return to the absorption column. A portion of a product stream formed from at least some of the bottom product of the dimethyl ether carbon dioxide distillation column is used as the return predominantly containing dimethyl ether. This bottom product preferably contains all or the predominant portion of the dimethyl ether prepared in the synthesis. The above-mentioned top gas, which can consist predominantly of carbon dioxide, is withdrawn from the top of the dimethyl ether carbon dioxide distillation column. The part of the top gas which is not used as a return for the dimethyl ether carbon dioxide distillation column can be recycled to provide synthesis gas and/or discharged from the plant in gaseous or condensed form in order either to be marketed as an additional product of value or to be discarded as waste. In the two latter cases, aftertreatment of the carbon dioxide can be provided, for example to set product specifications for purification or in the sense of, for example, legally prescribed, processing, for example for carbon capture and storage (CCS) applications. Particularly in cases in which the synthesis gas is provided using carbon dioxide, for example by an electrolysis method, the aforementioned recirculation of carbon dioxide to the provision of synthesis gas can advantageously be provided.

Advantageously, as explained in the introduction, not all the top gas freed from carbon dioxide is conveyed back from the absorption column to the synthesis. Some of it is thus advantageously discharged from the method as a purge stream. Since the top gas from the absorption column can still contain relevant amounts of dimethyl ether, it is particularly preferably provided to subject the purge stream to an additional wash with liquid carbon dioxide in order to convey the largest possible portions of the dimethyl ether present therein back into the dimethyl ether carbon dioxide distillation column. Some of the condensed top gas from the dimethyl ether carbon dioxide distillation column can advantageously be used for this wash. This has the additional advantage that no streams external to the method are used, which minimizes additional sources of impurities.

The cooling to the second temperature level is advantageously carried out by means of existing refrigerants, for example C2-refrigerants (for example liquid ethane, ethylene or an equivalent to achieve a corresponding temperature), DME or also carbon dioxide. If carried out, a preliminary stepwise cooling to intermediate temperature levels is carried out, for example, with water, water/glycol mixtures, a C3 refrigerant (for example liquid propane, propylene or an equivalent), DME or also carbon dioxide. The first temperature level is advantageously 10 to 50° C., in particular 20 to 40° C. The second temperature level is advantageously 0.5 to 20° C., in particular 1 to 10° C. above the melting temperature of carbon dioxide at the first pressure level.

The method proposed according to the invention proves to be economically significantly more favorable than conventional methods, and therefore the measures according to the invention achieve advantageous separation in comparison with separation methods as are known from the prior art. As a result of the recirculation according to the invention of the recycling stream depleted of carbon dioxide, the portion of shifted synthesis gas in the fresh input can be reduced, since an increase in the carbon dioxide portion in the product stream does not lead to a catalyst-damaging increase in the carbon dioxide portion in the input, as already explained in the introduction.

The present invention is suitable in particular for methods in which the product stream from the reactor used for the synthesis of dimethyl ether from synthesis gas is provided at a pressure level of 20 to 100 bar, in particular at a pressure level of 30 to 80 bar (the “first pressure level”). As already mentioned, the product stream is largely freed of methanol and water at this first pressure level using a further absorption column. The separation of methanol and/or water can thus take place under pressure; a previous expansion, which would then require energy-intensive pressurization again, is not necessary. The gas mixture obtained from the product stream is therefore not expanded after leaving the reactor for the synthesis of dimethyl ether and before the separation processing according to the invention. Another energy-intensive compression can therefore be dispensed with.

The method according to the invention can be used with product streams of a wide variety of compositions, as are obtained in the course of synthesis. Corresponding product streams contain, for example, 2 to 50 mole percent, in particular 5 to 30 mole percent dimethyl ether, 0.1 to 20 mole percent, in particular 0.7 to 10 mole percent methanol, 0.1 to 20 mole percent, in particular 0.8 to 10 mole percent water, 1 to 50 mole percent, in particular 3 to 30 mole percent carbon dioxide, 0.1 to 25 mole percent, in particular 1 to 11 mole percent carbon monoxide, and 5 to 90 mole percent, in particular 20 to 80 mole percent hydrogen. After water and methanol have been separated out, the gas mixture is preferably low in or free of water and methanol.

Corresponding product streams can further contain smaller portions of further components, for example methane, ethane, organic acids and higher alcohols, as mentioned. Corresponding mixtures are obtained in particular in methods in which a single-stage synthesis of dimethyl ether is carried out.

As already mentioned, in the context of the present invention, the dimethyl ether carbon dioxide distillation column is advantageously operated in such a way that a top gas which predominantly contains carbon dioxide and is drawn off from the top of the dimethyl ether carbon dioxide distillation column can be liquefied above the melting temperature of carbon dioxide. In this case, the dimethyl ether carbon dioxide distillation column is advantageously operated at a second pressure level which is below the first pressure level. The second pressure level is, for example, 10 to 40 bar, in particular 15 to 30 bar.

The bottom liquid can be withdrawn from the dimethyl ether carbon dioxide distillation column as a dimethyl-ether-rich stream which has a dimethyl ether content of at least 90 mole percent, in particular at least 95 mole percent or at least 98.5 mole percent. The respective contents depend on the operating conditions of the dimethyl ether carbon dioxide distillation column and its configuration. They can be adapted to the required specification (purity) of a product stream obtained accordingly. A dimethyl ether product which represents the actual target product of the method is also obtained from the bottom liquid of the dimethyl ether carbon dioxide distillation column.

As mentioned, the liquid return which is fed to the absorption column consists predominantly of dimethyl ether. For example, dimethyl ether contents of at least 90 mole percent, in particular at least 95 mole percent or at least 98.5 mole percent are advantageous.

The method makes it possible to reduce the portion of carbon dioxide in the last condensation stage by using the absorption column. The recycling stream formed from the top gas of the absorption column and predominantly containing carbon monoxide and hydrogen advantageously has a carbon dioxide content of at most 10, at most 7 mole percent, in particular at most 5 mole percent, at most 1 mole percent or at most 0.5 mole percent. Corresponding contents can also be obtained in the top stream of the absorption column.

To reduce losses of dimethyl ether, it is advantageously provided to condense the top gas of the absorption column at least in part and to convey it back into the top region of the absorption column.

A separation plant configured for the separation processing of the gas mixture explained several times likewise forms the subject matter of the present invention. The gas mixture is formed from a product stream of a reactor for the synthesis of dimethyl ether from synthesis gas. The separation plant has all the means which enable it to carry out the method explained above. Such a separation plant is in particular configured to carry out a method as explained above.

This separation plant, like a plant provided according to the invention for preparing dimethyl ether, benefits from the advantages explained above, to which reference is therefore expressly made.

The invention is explained in more detail with reference to the drawings, which show an embodiment of the invention in comparison with the prior art.

### BRIEF DESCRIPTION OF THE DRAWINGS

FIG. 1 is a schematic representation of a plant for producing dimethyl ether according to the prior art

FIG. 2 is a schematic representation of a plant for producing dimethyl ether according to an embodiment of the invention.

In the figures, elements corresponding to one another are indicated by identical reference signs and are not explained repeatedly for the sake of clarity.

### DETAILED DESCRIPTION OF THE DRAWINGS

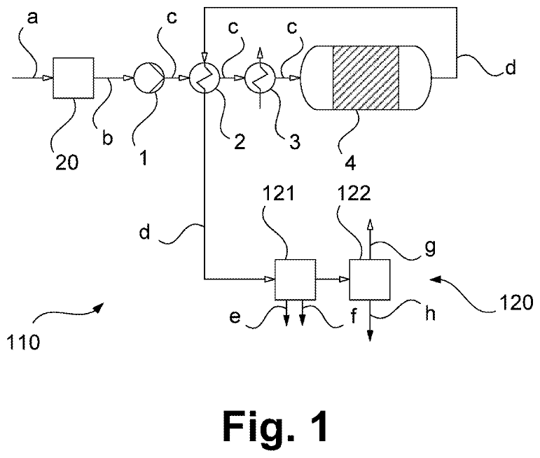

FIG. 1 schematically illustrates a plant for producing dimethyl ether according to the prior art, which is denoted as a whole by 110.

The plant 110 comprises a synthesis gas reactor 20, which is shown in a highly schematic manner here and which can be charged with a suitable input a, for example natural gas or biogas. A synthesis gas stream b can be withdrawn from the synthesis gas reactor 20.

The synthesis gas stream b can be pressurized, optionally after adding further streams, by means of a compressor 1. As a result, a pressure required for a subsequent single-stage dimethyl ether synthesis, for example a pressure of 20 to 80 bar, can be set.

A correspondingly compressed stream, now denoted by c, is conducted through a first heat exchanger 2 which can be heated with a product stream d of a reactor 4 for the synthesis of dimethyl ether (see below). The correspondingly heated stream, still denoted by c, has, for example, a temperature of 200 to 300° C. downstream of the first heat exchanger 2. The stream c is optionally conducted through a second heat exchanger 3, which is also referred to as a peak heater.

The stream further heated in the second heat exchanger 3, also referred to here as c, is fed into the reactor 4, which is formed as a tubular reactor, the reaction tubes of which are filled with a suitable catalyst for single-stage synthesis of dimethyl ether. The illustration in FIG. 1 is greatly simplified.

Typically, reactors 4 are arranged upright for the synthesis of dimethyl ether, a stream c being fed into the tube reactor 4 at the bottom. A stream d is withdrawn from the top of the reactor 4.

Due to the exothermic reaction in the tubular reactor 4, the stream d is at a still higher temperature. The stream d is conducted as a heating medium through the heat exchanger 2. As a result, it cools down to a temperature which is, for example, approximately 30° C. above the temperature of the stream c downstream of the compressor 1. The correspondingly cooled stream, still denoted by d, is fed to a conventional separation plant 120. In the separation plant 120, a methanol stream e and a water stream f are separated from the stream d, for example with intermediate expansion, cooling, recompression, etc. (not shown) in a step 121. The remaining residue is used to form the streams g and h, which may be, for example, a stream g enriched with carbon dioxide and a stream h enriched with dimethyl ether.

The composition of the streams g and h depends, inter alia, on the composition of the stream d and the specific configuration and operating parameters of the separation plant 120.

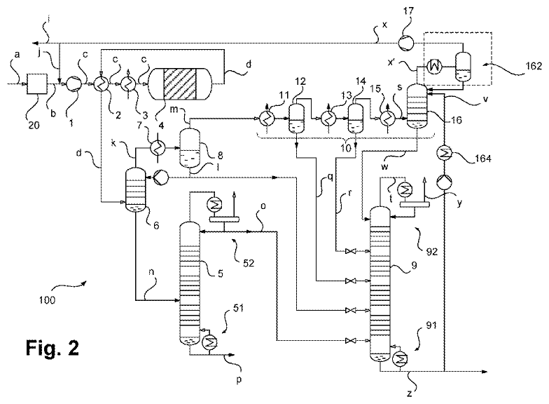

FIG. 2 illustrates a plant for producing dimethyl ether according to an embodiment of the invention. This is denoted as a whole by 100.

A first absorption column, which is used for separating out methanol and/or water, is denoted by 6 in FIG. 2. As already explained, an absorption column 6 differs from a distillation column such as the distillation column 5 in that it does not have a bottom evaporator, among other things. Vapors rising in the absorption column 6 are washed by a return provided at the top of the absorption column, so that the more volatile components are enriched at the top of the absorption column and the less volatile components are enriched at the bottom of the absorption column.

In the plant 100 shown in FIG. 2, the stream d is introduced into the absorption column 6. From the top of the absorption column 6, a top stream k is withdrawn and cooled in a heat exchanger 7 against a suitable refrigerant, for example cooling water. The correspondingly cooled stream k is transferred into a separator container 8, from the bottom of which a liquid stream l is withdrawn and at least partially fed to the absorption column 6 as a return by means of a pump (without designation).

If the stream d in the example shown also contains methanol, water, carbon dioxide, carbon monoxide and hydrogen (as well as traces of other compounds as explained above) in addition to dimethyl ether, then dimethyl ether, carbon dioxide, carbon monoxide and hydrogen pass therefrom into the top stream k, due to the backwash explained. As a result of suitable cooling in the heat exchanger 7 and corresponding deposition conditions in the separator container 8, a bottom product is deposited in the separator container 8, which bottom product consists substantially of dimethyl ether and carbon dioxide (possibly with traces of methanol).

From the top of the separator container 8, a stream m can be drawn off in gaseous form, which also contains dimethyl ether in addition to carbon dioxide, carbon monoxide and hydrogen. The stream m subsequently undergoes sequential cooling and condensation, as explained below. The portion of the stream l that is not fed as a liquid return to the absorption column 6 is fed, like the condensates obtained during the sequential cooling and condensation of the stream m, into a distillation column 9, referred to here as dimethyl ether carbon dioxide distillation column 9.

It is expressly emphasized that the specific provision of the stream k obtained from the product stream d does not have to take place in the manner shown. Other possibilities for separating out water and/or methanol can also be used, as long as they lead to the production of a gas mixture at the aforementioned first pressure level and the first temperature level and with the stated contents of the individual components.

It is also possible, for example, to dispense with the distillation column 5 and to separate water from the stream n in another way. In particular, the water is thus fed into the dimethyl ether carbon dioxide distillation column 9. In such cases, the water may be separated from the dimethyl ether product z in a plant component downstream of the dimethyl ether carbon dioxide distillation column 9.

A liquid stream n is withdrawn from the bottom of the absorption column 6 and fed at a suitable level into the distillation column 5, which is operated with a bottom evaporator 51 and a top condenser 52. In the example shown, the stream n contains the predominant portion of the water and methanol contained in the stream d.

The bottom evaporator 51 and the top condenser 52 are operated with suitable heating or cooling media, preferably present in a corresponding plant. In the bottom evaporator 51, a liquid stream withdrawn from the bottom of the distillation column 5 is partially evaporated and fed into a lower region of the distillation column 5. A non-evaporated portion can be drawn off as stream p.

From the top of the distillation column 5, a gaseous stream is drawn off, partially liquefied in the top condenser 52 of the distillation column 5, and fed back into an upper region of the distillation column 5 as a liquid return. A portion o remaining in gaseous form is drawn off.

In the distillation column 5, a stream (stream o) containing substantially dimethyl ether and carbon dioxide and a stream (stream p) containing substantially methanol and water are thus formed from the stream n, which substantially still contains water, methanol, hydrogen, dimethyl ether and carbon dioxide. The stream o can be conveyed back into the separation process at a suitable point. The stream p can be used elsewhere. Deposited water can also be conducted to a wastewater treatment or a degassing process.

The distillation column 5 can also be operated in such a way that substantially no water, but methanol, passes into the top product o in a non-negligible amount. This is advantageous, for example, when the dimethyl ether is to be used for purposes in which methanol does not interfere with use. Thus, a higher yield can be achieved in relation to the synthesis gas used.

The return quantity and bottom number of the absorption column 6 can be optimized in such a way that the smallest possible amount of a corresponding bottom product n is produced.

Advantageously, the return which is applied as a portion of the stream l to the absorption column 6 is set such that the methanol and/or water content in the stream k is minimized. The composition of the stream m resulting in this way is such that the disadvantages explained at the outset can no longer arise in the cooling and condensation sequence to which the stream m is subjected.

The steps already mentioned several times for further treatment of the stream m are indicated here as a whole with 10. The stream m is firstly fed to a heat exchanger 11 and then fed into a separator container 12. The cooling in the heat exchanger 11 is carried out in such a way that a first condensate q is deposited in the separator container 12. A portion remaining in gaseous form in the separator container 12 is fed to a heat exchanger 13 and then fed into a further separator container 14. A condensate, referred to here as r, is also obtained there.

The condensates q and r are fed together with the portion of the stream l not conveyed back to the absorption column 6 into the above-mentioned dimethyl ether carbon dioxide distillation column 9, which is operated as explained below. A portion remaining in gaseous form at the top of the separator container 14 is cooled in a further heat exchanger 15. This stream, referred to here as s, at the “second” temperature level explained several times just above the melting point of carbon dioxide (at the prevailing pressure) downstream of the heat exchanger 15. In contrast, the temperature of the stream m upstream of the heat exchanger 11 (i.e., the “first” temperature level) is for example 35° C. The correspondingly cooled stream s is transferred into an absorption column 16, which can be operated according to the invention.

The invention can also be used in a highly simplified arrangement, for example in a single-stage cooling system which is connected downstream of the separation of methanol and water. However, a portion of the gas mixture remaining in gaseous form at the second temperature level is always washed in an absorption column 16 with a return predominantly containing dimethyl ether, as explained below. The return predominantly containing dimethyl ether is here formed from a liquid portion of the gas mixture deposited during cooling.

In the example shown, the stream s also contains dimethyl ether, carbon dioxide, carbon monoxide and hydrogen, i.e., in addition to dimethyl ether and carbon dioxide here two components which boil more easily than carbon dioxide. Using a liquid return v which is rich in dimethyl ether and is formed from some of a condensate z obtained from a bottom stream from a bottom liquid of the dimethyl ether carbon dioxide distillation column 9, a mixture of dimethyl ether and carbon dioxide is deposited in the bottom of the absorption column 16 and drawn off in the form of the bottom product w. The bottom product w can likewise be fed into the dimethyl ether carbon dioxide distillation column 9. At the top of the absorption column 16, however, a top product x is drawn off, which consists substantially of carbon monoxide and hydrogen and is low in or preferably free of carbon dioxide. This is, if appropriate after appropriate compression in a compressor 17, added at least in part as a recycling stream j to the stream b.

A further part of the top product x depleted of carbon dioxide from the absorption column 16 can be discharged from the method as a purge stream i.

The purge stream i can be subjected to an additional wash with liquid carbon dioxide in order to convey the largest possible portions of the dimethyl ether contained therein back into the dimethyl ether carbon dioxide distillation column 9. Some of the condensed top gas t from the dimethyl ether carbon dioxide distillation column 9 can advantageously be used for this wash. This makes it possible to reduce the losses of dimethyl ether, which otherwise would result via the discharge of the purge stream i. However, since only the purge stream i is subjected to this additional wash, no additional carbon dioxide is introduced into the recycling stream j as a result. Dimethyl ether contained in the recycling stream j is thus conveyed back through the reactor 4 into the separation cycle and thus remains largely retained.

In some embodiments, the top gas x′ of the absorption column 16 can be condensed in an optional top condenser 162 and used with the stream v as a return to the absorption column 16. This reduces the portion of dimethyl ether in the top product x, which leads to an improved reaction equilibrium and reduced loss via the purge stream i.

As mentioned, the portion of the stream l not conveyed back into the absorption column 6 and the streams q and r and the bottom product w are fed into the dimethyl ether carbon dioxide distillation column 9. Since these have different contents of dimethyl ether and carbon dioxide (traces of carbon monoxide and hydrogen are also present in dissolved form), they are fed into the dimethyl ether carbon dioxide distillation column 9 at different heights, for which purpose suitable valves (without designation) are provided.

The dimethyl ether carbon dioxide distillation column 9 is also operated with a bottom evaporator 91 and a top condenser 92. A top stream t formed from a top gas of the dimethyl ether carbon dioxide distillation column 9 is at least partially liquefied in the top condenser 92 using a heat exchanger operated with a suitable refrigerant and fed as a liquid return to the dimethyl ether carbon dioxide distillation column 9. A further portion is used to form a further stream y, which can be used elsewhere.

A liquid stream z is withdrawn from the bottom of the dimethyl ether carbon dioxide distillation column 9, which here consists substantially of dimethyl ether, but is in particular free of or low in carbon dioxide. Some of this bottom stream z is used to form the return v for the absorption column 16. In addition, the dimethyl ether product is formed from the bottom stream z.

The condensed top stream o of the distillation column 5 is also suitable as the return v, since it is substantially free of carbon dioxide and rich in dimethyl ether and possibly methanol.

If water is separated out only downstream of the dimethyl ether carbon dioxide distillation column 9, as described above with respect to an embodiment, it is expedient to use a substantially water-free side stream of the dimethyl ether carbon dioxide distillation column 9 instead of its bottom product z as the return v for the absorption column 16. In this case, the side stream is selected such that it contains less than 10 mass percent, in particular less than 5 mass percent carbon dioxide.

The return v is cooled before being fed to the absorption column 16, typically to a temperature level which is somewhat above the freezing point of carbon dioxide at the selected conditions, for example to a temperature level in the range from −30° C. to −49° C. For this purpose, a heat exchanger 164 can be provided, for example.

1. A method for preparing dimethyl ether (DME) from synthesis gas, wherein an input, which is formed using shifted and/or non-shifted synthesis gas, undergoes a catalytic conversion, thereby forming a product stream, wherein the product stream undergoes a first separation, wherein a gas mixture is formed by at least partial separation of methanol and/or water from the product stream, and the gas mixture is partially condensed at a first pressure level by means of cooling from a first to a second temperature level, wherein a portion of the gas mixture remaining in gaseous form at the second temperature level is washed in an absorption column with a return predominantly containing dimethyl ether, wherein the return predominantly containing dimethyl ether is formed at least partially from a portion of the gas mixture condensed during cooling, and wherein the gaseous portion of the gas mixture not washed out in the absorption column is at least partially transferred into the input, and a dimethyl ether product is formed using the portion of the gas mixture condensed during cooling.

2. The method according to claim 1, in which the gas mixture is cooled to the second temperature level via multiple intermediate temperature levels, wherein multiple condensates are deposited.

3. The method according to claim 1, in which the portion of the gas mixture remaining in gaseous form at the second temperature level is washed in the absorption column with the return predominantly containing dimethyl ether, thereby obtaining a top product and a bottom product, wherein the return predominantly containing dimethyl ether is formed at least partially from the bottom product and/or partially from the top product.

4. The method according to claim 1, in which the return predominantly containing dimethyl ether is formed using a dimethyl ether carbon dioxide distillation column.

5. The method according to claim 4, in which the dimethyl ether carbon dioxide distillation column is operated in such a way that a top gas predominantly containing carbon dioxide forms at the top, and a bottom liquid enriched with dimethyl ether forms at the bottom.

6. The method according to claim 5, in which some of the dimethyl ether product which is formed from at least some of the bottom liquid of the dimethyl ether carbon dioxide distillation column is used as the return predominantly containing dimethyl ether.

7. The method according to claim 5, in which at least some of the bottom liquid of the dimethyl ether carbon dioxide distillation column is drawn off as a dimethyl ether product having a dimethyl ether content of more than 90 mole percent, in particular more than 95 mole percent, in particular more than 98.5 mole percent.

8. The method according to claim 4, in which the dimethyl ether carbon dioxide distillation column is operated at a second pressure level below the first pressure level.

9. The method according to claim 1, in which the return predominantly containing dimethyl ether has a carbon dioxide content of at most 5 percent by weight, in particular at most 2 percent by weight, at most 1 percent by weight, at most 0.5 percent by weight or at most 0.1 percent by weight.

10. The method according to claim 1, wherein a portion, not transferred into the input, of the gaseous portion of the gas mixture not washed out is discharged in part from the method as a purge stream, wherein the purge stream is subjected to a wash with at least partially liquid carbon dioxide, which is provided in particular using the dimethyl ether carbon dioxide distillation column, thereby obtaining a wash stream, and the wash stream is at least partially conveyed back into the dimethyl ether carbon dioxide distillation column.

11. The method according to claim 1, in which the first temperature level is 10 to 50° C., in particular 20 to 40° C., and/or in which the second temperature level is 0.5 to 20° C., in particular 1 to 10° C. above the melting temperature of carbon dioxide at the first pressure level, and/or in which the first pressure level is 20 to 100 bar, in particular 30 to 80 bar.

12. The method according to claim 1, wherein the synthesis gas is transferred into the input via the absorption column together with the gaseous portion of the gas mixture not washed out.

13. The method according to claim 1, wherein a top gas of the absorption column is at least partially condensed and the condensed portion of the top gas x′ is at least partially conveyed back into the top region of the absorption column.

14. A separation plant which is configured for the separation processing of a gas mixture which can be formed from a product stream of a reactor for the synthesis of dimethyl ether from synthesis gas, and which contains at least dimethyl ether, carbon dioxide and at least one further component which boils more easily than carbon dioxide, with means which are configured to provide the gas mixture while at least partially separating methanol and/or water from the product stream and to cool it at a first pressure level from a first to a second temperature level and to wash a portion of the gas mixture remaining in gaseous form at the second temperature level in an absorption column with a return predominantly containing dimethyl ether, wherein means are further provided which are configured to form the return predominantly containing dimethyl ether at least partially from a portion of the gas mixture condensed during cooling.

15. A plant for preparing dimethyl ether, comprising at least one reactor configured for the synthesis of dimethyl ether from synthesis gas and a separation plant according to claim 14.
